(19) United States
(12) Patent Application Publication
(10) Pub. No.: US 2015/0147818 A1
(43) Pub. Date: May 28, 2015

(54) SENSOR FOR DETECTING EXPLOSIVE, AND PREPARATION METHOD THEREOF

(71) Applicant: POSTECH ACADEMY-INDUSTRY FOUNDATION, Pohang-si (KR)

(72) Inventors: Sungjee Kim, Pohang-si (KR); Nayoun Won, Seoul (KR); Jungheon Kwag, Gimhae-si (KR); Joonhyuck Park, Pohang-si (KR); Ho Jin, Seosan-si (KR)

(21) Appl. No.: 14/404,297

(22) PCT Filed: May 6, 2013
(86) PCT No.: PCT/KR2013/003887

§ 371 Date: Nov. 26, 2014

(30) Foreign Application Priority Data
May 30, 2012 (KR) 10-2012-0057344

(51) Int. Cl. G01N 33/22; G01N 21/64

(52) U.S. Cl. CPC G01N 33/227; G01N 21/6408; G01N 2201/08; G01N 2201/061; G01J 3/4406
USPC 436/110; 422/69

(57) ABSTRACT

The present invention relates to a sensor capable of detecting an aromatic nitro compound explosive, and a preparation method thereof, and more specifically, to a nanosensor system, and a detection method using the same, wherein a quantum dot-based sensor for detecting an aromatic nitro compound explosive can conveniently detect an aromatic nitro compound explosive with high sensitivity on the basis of a change in energy transfer between quantum dots. The method for detecting an explosive of the present invention makes an explosive come in contact with a quantum dot thin film to which an explosive can combine, and measures a change in fluorescence wavelength, thereby sensing an explosive. According to the present invention, the method for detecting an explosive on the basis of quantum dots uses a change in fluorescence wavelength which is unlike a known detection method using the change in quantum dot fluorescence intensity, and thus is not sensitive to a change in surroundings, can carry out rapid detection, and can detect even a low concentration of explosives with high sensitivity. Therefore, the present invention is expected to be extensively commercialized.

### TECHNICAL FIELD

The present invention relates to a sensor for detecting a nitroaromatic compound explosive and a method of manufacturing the same, and more particularly, to a nanosensor system for detecting a nitroaromatic explosive wherein a quantum dot-based sensor enables a nitroaromatic explosive to be simply detected at high sensitivity based on changes in energy transfer between quantum dots, and to a detection method using the same.

### BACKGROUND ART

Typical compounds useful as explosives include nitroaromatic chemicals such as trinitrotoluene (TNT) or dinitrotoluene (DNT). A variety of methods for detecting such chemicals to sense explosives have been developed. Research and development into methods of detecting chemicals contained in explosives using ion mobility spectroscopy or neutron detection is ongoing, but these methods are problematic because of relatively long detection time and high cost, compared to when using biosensors.

Sensors using fluorescence allow a measurement system to be simplified and may exhibit high sensitivity compared to other physical changes such as light absorption, and thus are mainly applied as representative chemical sensors.

Conventional detections are carried out by a quantum point-based sensor that shows fluorescence decay when binding with explosives. When a molecule having a terminal primary amine group is introduced onto the quantum dot surface, it is known that the primary amine group and TNT are formed into a Meisenheimer complex or that TNT anions are attracted to the positively charged amine ligand due to acid-base interactions between amine and TNT. When an explosive including a nitro group such as TNT binds to the quantum dot surface in this way, fluorescence is known to decrease by moving electrons to the electron-deficient nitro group from the quantum dots.

To increase detection sensitivity, an acceptor that specifically binds with an explosive is introduced to the quantum dot surface, after which a TNT derivative with a quencher is attached thereto, so that quantum dot fluorescence is quenched. Then, fluorescence is observed to increase as TNT replaces the derivative. However, a sensor for measuring fluorescence intensity is undesirably sensitive to changes in the ambient environment including temperature, pH, ion intensity, etc.

Therefore, there is required a fluorescence sensor, which may measure changes in fluorescence to thereby allow for a simple measurement system and is less sensitive to the ambient environment including temperature, pH and ion intensity while exhibiting high detection sensitivity compared to other physical changes such as light absorption.

### DISCLOSURE

### Technical Problem

Accordingly, an object of the present invention is to provide a fluorescence sensor, which may measure changes in fluorescence to thereby allow for a simple measurement system and is less sensitive to the ambient environment including temperature, pH and ion intensity while exhibiting high detection sensitivity compared to other physical changes such as light absorption; and a method of manufacturing the same.

Another object of the present invention is to provide a fluorescence sensor using changes in fluorescence wavelength, instead of fluorescence intensity, so as to increase explosive detection sensitivity with less influence to changes in the ambient environment; and a method of manufacturing the same.

### Technical Solution

According to the present invention, an explosive sensor functions to sense an explosive using a change in fluorescence wavelength by bringing an explosive-bindable quantum dot thin film into contact with an explosive.

In an aspect, the explosive sensor according to the present invention is a sensor for detecting an explosive, comprising: a light source; a substrate having an explosive-bindable quantum dot thin film; and a fluorescence spectrometer for measuring a change in fluorescence of quantum dots.

In another aspect, a method of detecting an explosive according to the present invention includes bringing an explosive-bindable quantum dot thin film into contact with an explosive, and detecting the explosive using a change in fluorescence wavelength.

In still another aspect, a method of detecting an explosive according to the present invention includes bringing a sample into contact with a substrate coated with explosive-bindable quantum dots, and measuring a change in fluorescence of the quantum dots.

In the present invention, the explosive-bindable quantum dot thin film comprises quantum dot nanoparticles having a molecule able to bind with an explosive on the surface thereof or is a thin film including quantum dot nanoparticles.

In the present invention, the quantum dot thin film is preferably formed so that a difference in fluorescence wavelength from a quantum dot solution is 10 nm or more, preferably 30 nm or more, and more preferably 50 nm or more.

In the present invention, the explosive-bindable quantum dot thin film is not theoretically limited, but is configured such that a distance between quantum dots is short, thus causing energy transfer between the quantum dots, thereby showing long-wavelength fluorescence compared to the quantum dot solution. Also, when an explosive binds to such a quantum dot thin film, the distance between the quantum dots may increase or the energy transfer between the quantum dots may be impaired, so that the wavelength of the quantum dot thin film is shifted to a short wavelength.

As used herein, ‘quantum dot solution’ means that quantum dots, which constitute the quantum dot thin film or are incorporated in the quantum dot thin film, are dispersed or dissolved in a liquid such as water or an organic solvent.

In the present invention, the change in fluorescence wavelength of the quantum dot thin film may be accompanied by a change in fluorescence intensity, for example, a decrease or an increase in fluorescence intensity.

In the present invention, the quantum dot thin film may be provided in the form of a thin film by applying the quantum dot solution on a substrate and then drying it. A process of forming a thin film may include, but is not limited to, for example, drop-casting, spin-casting, or dip-coating.

In an embodiment of the present invention, the quantum dot thin film is preferably formed to a thickness of about 0.1˜100 μm in order to effectively observe energy transfer between quantum dots.

In an embodiment of the present invention, the quantum dot thin film may have a concentration of about 0.1˜10 pmol/cm².

In the present invention, contact between the quantum dot thin film and the explosive is preferably performed in such a manner that a liquid sample is added dropwise onto the thin film coated with quantum dots.

In the present invention, in order to evaluate the change in fluorescence wavelength of the quantum dot thin film due to the explosive, a light source for exciting quantum dots is required. For visible light fluorescence, a change in fluorescence wavelength may be observed with the naked eye or using a fluorescence microscope. For quantitative analysis, a fluorescence spectrum may be obtained with a spectrometer connected by an optical fiber.

Since a change in the fluorescence wavelength of the quantum dot thin film is proportional to the concentration of the explosive, the concentration of the explosive may be measured while detecting the explosive.

In the present invention, the quantum dot is not particularly limited so long as a change in fluorescence occurs due to binding with the explosive, but is preferably composed of a semiconductor nanoparticle to which a molecule able to bind with an explosive is introduced.

In the present invention, the nanoparticle refers to a nanoparticle having a diameter of less than 1000 nm. In some embodiments, the nanoparticle may have a diameter of less than 300 nm based on the definition by the National Science Foundation. In some embodiments, the nanoparticle may have a diameter of less than 100 nm based on the definition by the National Institutes of Health.

In the present invention, the nanoparticle may comprise a single nanoparticle, and many nanoparticles may be provided in the form of a single nanoparticle by aggregation. The nanoparticle may be a high-density nanoparticle, the inside of which is compact, or may be provided in the form of a nanoparticle having a partition or a space therein. In an embodiment of the present invention, the nanoparticle may be provided in the form of a monolayer or a multilayer.

In the present invention, the molecule may be a monomer, an oligomer such as a dimer or trimer, or a polymer compound. Preferably, the length of the molecule is shorter than the outer diameter of the nanoparticle, so that the molecule is not completely provided around the nanoparticle and is linked in a manner that extends outwards from the center of the particle under the dispersed condition, whereby the part to bind with an explosive may be distributed on the outermost surface of the nanoparticle.

In the present invention, the molecule includes an attachment domain that is strongly linked to the nanoparticle surface at one end thereof, a functionality domain able to bind with a nitroaromatic explosive at the other end thereof, and an intermediate connection domain between the attachment domain and the functionality domain.

In an embodiment of the present invention, the attachment domain is capable of being strongly linked to the nanoparticle surface, and may be used without limitation so long as it may form stable bonding with the surface of the nanoparticle. Examples thereof may include, but are not limited to, a thiol group (—SH), an amine group (—NH₂, —NH), a phosphonate group (—PO₃H), a phosphide group (—P), a phosphine oxide group (—P═O), a carboxyl group (—COOH), a hydroxyl group (—OH), an imidazole group, and a diol group.

In an embodiment of the present invention, the functionality domain is located at the terminal opposite the attachment domain of the surface molecule, and refers to a domain able to bind with a nitroaromatic explosive such as TNT. It includes an amine group, a peptide or an antibody, which is able to bind with TNT, but is not limited thereto.

In an embodiment of the present invention, the connection domain is provided by strong covalent bonding to connect the attachment domain and the functionality domain to each other so as to form a single molecule. It may function to introduce different functionality domains with a predetermined attachment domain, or to introduce different attachment domains to a predetermined functionality domain. Hence, this connection domain may include a variety of functional groups for connection of desired molecules. The usable connection domain may include amide bonding (—CONH—), carbon bonding (—(CH₂)ₙ—), polyethyleneglycol (—(CH₂CH₂O)ₙ—), or triazole, wherein n is preferably an integer of 1˜100 and more preferably 1˜20, but is not limited thereto.

In the present invention, quantum dot nanoparticles bindable with explosives are prepared into a quantum dot solution by the molecule-ligand substitution under the condition that they are dispersed in water and/or an organic solvent, and then the quantum dot solution is applied and dried, thus forming a thin film.

The aspect of the present invention addresses a sensor for detecting an explosive, comprising: a light source; a substrate having an explosive-bindable quantum dot thin film; and a fluorescence spectrometer for measuring a change in fluorescence of quantum dots.

In the present invention, the fluorescence of the quantum dots is transferred to a fluorescence spectrometer via an optical fiber so as to be analyzed, and the quantum dots have a molecule able to bind with the explosive on the surface thereof.

In order to measure a trace amount of a sample, a fluorescence microscope may be further provided, and the substrate may be a glass substrate so that a fluorescence change may be observed with the fluorescence microscope. However, since fluorescence is measured, a silicon wafer may be used, in addition to the glass substrate, or the use of an opaque substrate is possible.

In an embodiment of the present invention, the sensor may detect an explosive at 10 ppt or more.

### Advantageous Effects

According to the present invention, a quantum dot-based explosive detection method employs changes in fluorescence wavelength, unlike conventional detection methods using changes in the fluorescence intensity of quantum dots. Hence, this method is not sensitive to changes in the ambient environment, enables rapid detection, and can detect low-concentration explosives at high sensitivity. Therefore, extensive commercialization thereof is expected in the future.

### DESCRIPTION OF DRAWINGS

FIG. 1 schematically illustrates nanoparticle surface modification where a nanoparticle synthesized in an organic solvent is surface-substituted with a molecule able to bind with an explosive;

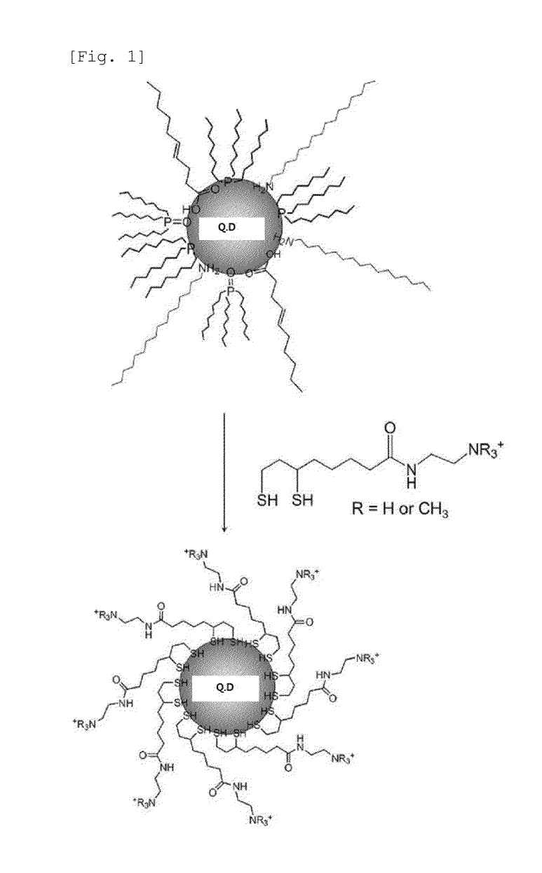

FIG. 2 illustrates fluorescence spectra for a quantum dot solution (black) dispersed in an aqueous solution and a quantum dot thin film (red) obtained by subjecting the quantum dot solution to drop-casting on a glass substrate and drying it, wherein the thin film formed from the quantum dot solution has a short distance between quantum dots to thus cause energy transfer, so that the fluorescence wavelength is shifted to a long wavelength;

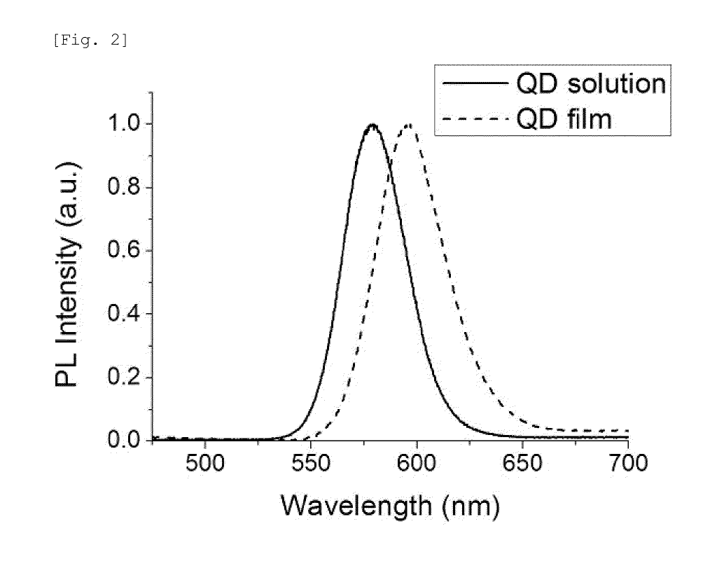

FIG. 3 schematically illustrates an explosive detection process using a quantum dot thin film, wherein a light source for exciting a quantum dot is applied and a fluorescence spectrometer connected by an optical fiber is provided to obtain a fluorescence spectrum, thus measuring a change in fluorescence wavelength of the quantum dot depending on whether the explosive is present or not;

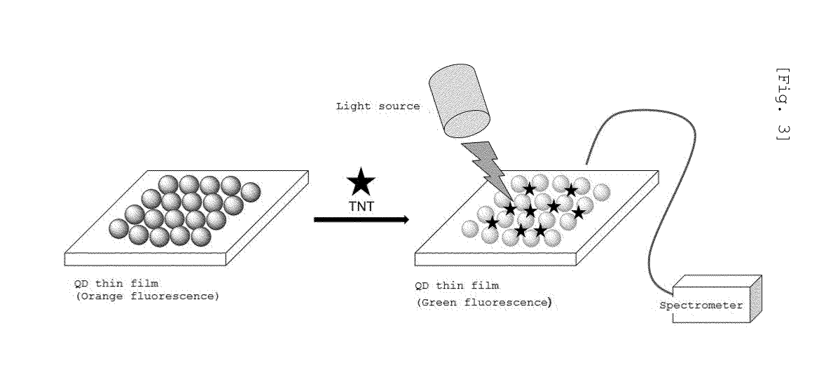

FIG. 4 illustrates fluorescence microscope images of a quantum dot thin film (left) and a quantum dot thin film containing 2 μL of 10 μM TNT dissolved in water (right), wherein the fluorescence of the quantum dot is shifted to a short wavelength in the presence of TNT;

FIG. 5 illustrates a change in fluorescence spectrum (left) of the quantum dot thin film depending on the concentration of TNT added to the quantum dot thin film, and the extent of shift of fluorescence peak (right), wherein the amount of TNT may be quantitatively analyzed by the extent of shift of fluorescence peak, and the detection threshold is about 10 ppt or less; and

FIG. 6 illustrates fluorescence spectra when water and toluene are added to the quantum dot thin film, wherein there is no change in fluorescence wavelength of the quantum dot thin film.

### MODE FOR INVENTION

### Example 1

### Synthesis of CdSe/CdS/ZnS (Core/Shell/Shell) Quantum Dots

Synthesis of quantum dots disclosed herein is merely illustrative and is not construed as limiting the present invention.

For quantum dots having high fluorescence efficiency, CdSe quantum dots were synthesized via high-temperature pyrolysis in an organic solvent and then covered with CdS/ZnS shells, thus synthesizing quantum dots having a structure of CdSe/CdS/ZnS (core/shell/shell).

Specifically, cadmium selenide (CdSe) quantum dots were synthesized via modification of the method reported by Yu and Peng (W. W. Yu and X. Peng. Angew. Chem. Int. Edit. 2002, 41, 2368-2371). In a septum vial, 0.75 g (2.4 mmol) of cadmium acetate and 1.8 mL (6.0 mmol) of oleic acid were placed and dissolved at 100° C. in a vacuum. The completely dissolved cadmium acetate was cooled to room temperature, and then mixed with a solution of 0.47 g of selenium in 6 mL of trioctylphosphine (TOP). 15 mL of octadecene and 4 mL (12 mmol) of oleylamine were placed in a 50 mL 3-neck round-bottom flask, and then heated to 315° C. in the presence of nitrogen gas. At a raised temperature, the mixture of cadmium and selenium was rapidly injected into a reactor. After 30 sec, the heating mantle was removed, and the reaction solution was cooled to room temperature. The synthesized cadmium selenide quantum dots were diluted with hexane, and nanocrystals were precipitated with an excess of methanol using a centrifuge to remove the organic reaction residue.

Synthesis of CdSe/CdS/ZnS (core/shell/shell) quantum dots by sequentially coating the synthesized CdSe quantum dots with CdS/ZnS shells was performed with reference to the method reported by Dabbousi et al. (B. O. Dabbousi et al., J. Phys. Chem. B 1997, 101, 9463-9475). In a 50 mL 3-neck round-bottom flask, 15 mL of octadecene was placed, and a CdSe solution (1.70×10⁻⁴ mmol) dispersed in 2 mL of hexane was injected at 60° C. in the presence of nitrogen gas. The hexane was removed in a vacuum. The temperature was adjusted to 120° C., and a Cd/S precursor containing 5 mL of TOP and 24.7 μL of bis(trimethylsilyl)sulfide was added using a syringe pump to a solution of 38 mg of cadmium acetate in 95 μL of oleic acid, and stirred for 30 min. The temperature was adjusted to 140° C., and a Zn/S precursor containing 44.8 μL of diethylzinc and 82.1 μL of bis(trimethylsilyl)sulfide dissolved in 10 mL of TOP was added using a syringe pump and stirred for 30 min. After completion of the reaction, the CdSe/CdS/ZnS (core/shell/shell) quantum dots were precipitated with methanol as in the CdSe quantum dots.

### Example 2

### Synthesis of Ligand on Nanoparticle Surface with Terminal Amine

A ligand on the quantum dot surface was synthesized by linking N,N-dimethylethylendiamine to (±)-α-lipoic acid. Then, (±)-α-lipoic acid (20 mmol) and 1,1′-carbonyldiimidazole (26 mmol) were dissolved in 30 mL of anhydrous chloroform and stirred at room temperature for 20 min in the presence of nitrogen gas. This solution was added dropwise into a flask containing N,N-dimethylethylendiamine (100 mmol) in an ice bath in the presence of nitrogen gas, and stirred for 2 hr. The product (LA-N(CH₃)₂) was washed three times with a 10% NaCl aqueous solution (80 mL) and two times with a 10 mM NaOH aqueous solution (80 mL), and then dewatered with magnesium sulfate.

### Example 3

### Surface Modification of Quantum Dots

The surface of the CdSe/CdS/ZnS quantum dots synthesized in Example 1 was modified with the LA-N(CH₃)₂ ligand synthesized in Example 2. LA-N(CH₃)₂ (0.1 mmol) was dispersed in 2 mL of chloroform, and then dispersed in 2 mL of water with the addition of an aqueous solution at about pH 4. The aqueous solution containing dispersed LA-N(CH₃)₂ was added with NaBH₄ (0.2 mmol), so that disulfide bonding of LA-N(CH₃)₂ was reduced, thus forming dihydrolipoic acid-tertiary amine (DHLA-N (CH₃)₂). The pH value was raised to about 10, and DHLA-N(CH₃)₂ was dispersed in chloroform, added with CdSe/CdS/ZnS quantum dots (1 nmol) dispersed in chloroform, and stirred at 60° C. for about 3 hr in the presence of nitrogen gas. The pH value was lowered to about 5, and the surface-modified quantum dots were dispersed in the aqueous solution and dialyzed using a 50,000 centrifugal filter, thus removing the surplus ligand.

### Example 4

### Explosive Detection Using Quantum Dot Thin Film

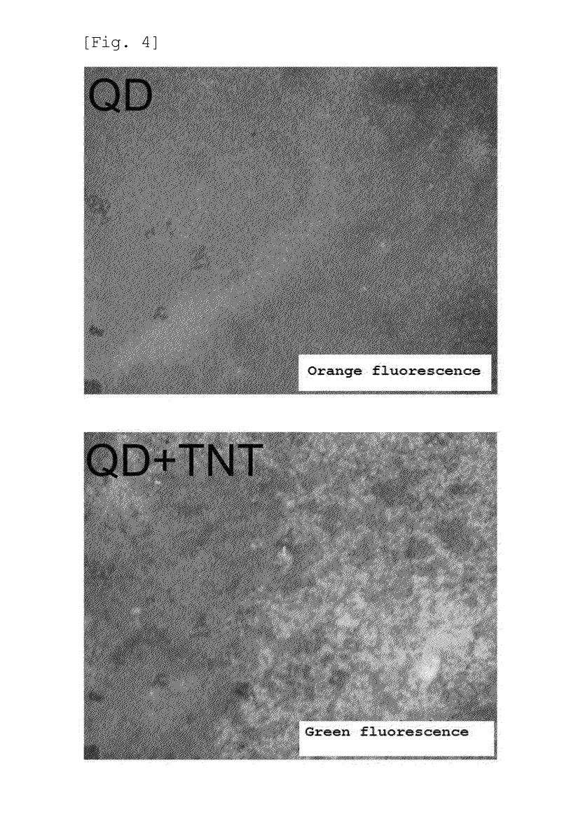

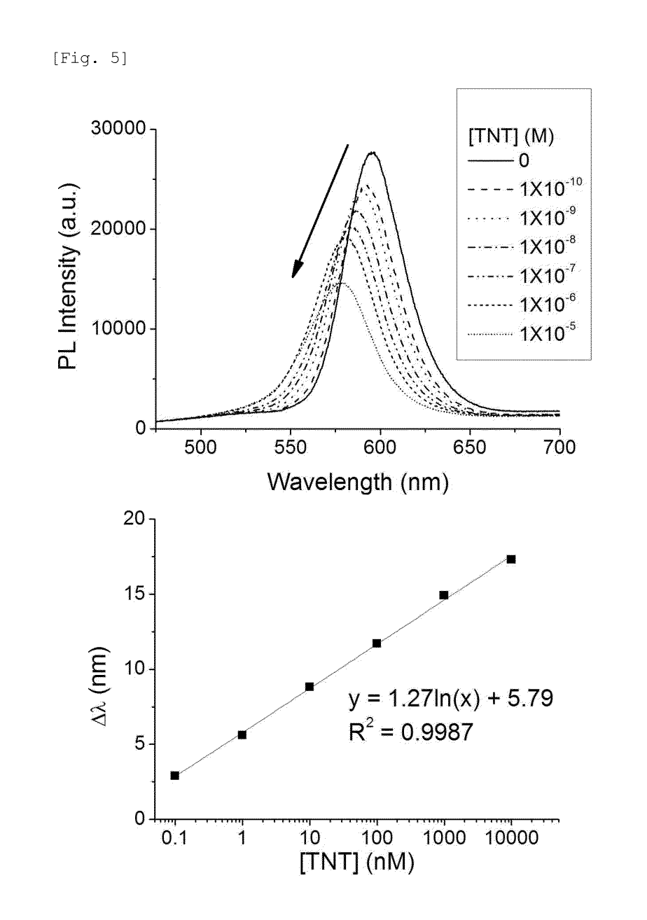

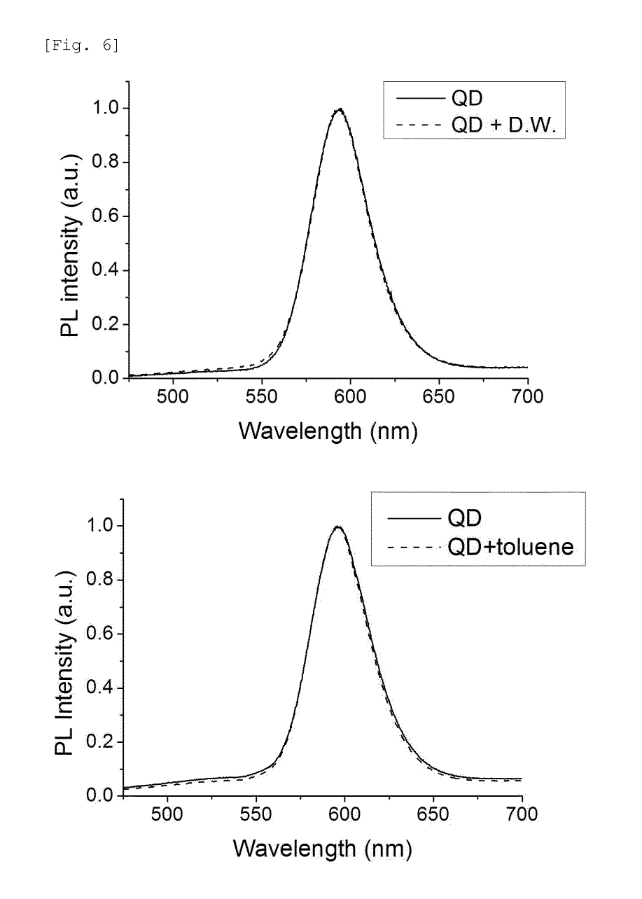

The quantum dots of Example 3 dispersed in an aqueous solution were diluted to 100 nM, drop-cast on a glass substrate and then naturally dried. The glass substrate was placed on a fluorescence microscope (Zeiss, Axioplan2), and fluorescence of the quantum dots was observed with a 20× objective lens, a 325 to 375 nm transmission light source filter and a 420 nm or more transmission fluorescence filter. FIG. 4 illustrates fluorescence microscope images for the quantum dot thin film (left) and the quantum dot thin film containing 2 μL of 10 μM TNT dissolved in water (right). When TNT was added, fluorescence of the quantum dots was shifted to a short wavelength. After addition of TNT, a change in the fluorescence wavelength was immediately initiated, and all the fluorescence measurements were performed within 5 min after addition of TNT. To evaluate the fluorescence wavelength of the quantum dots, the fluorescence spectrum was obtained with a fluorescence spectrometer (Horiba JobinYvon, Fluorlog3) by connecting an optical fiber to the CCD of the microscope. The results are shown in FIG. 5. As the concentration of TNT was higher, the wavelength of the quantum dot thin film was shifted to a short wavelength, and the detection threshold was about 10 ppt or less. In order to evaluate the effect of the solvent, when water was added to the quantum dot thin film, there was no change in fluorescence wavelength of the quantum dots, as shown in the left of FIG. 6. Also, the addition of toluene as a control of TNT to the quantum dot thin film resulted in no change in fluorescence wavelength, as shown in the right of FIG. 6.

1. A method of detecting an explosive, comprising bringing an explosive-bindable quantum dot thin film into contact with an explosive, and measuring a change in fluorescence wavelength.

2. The method of claim 1, comprising bringing a sample into contact with a substrate coated with an explosive-bindable quantum dot, and measuring a change in fluorescence of the quantum dot.

3. The method of claim 1, wherein the change in fluorescence wavelength is a fluorescence change from a long wavelength to a short wavelength.

4. The method of claim 1, wherein the change in fluorescence wavelength is accompanied by a change in fluorescence intensity.

5. The method of claim 1, wherein a concentration of the explosive is measured using the change in fluorescence wavelength.

6. The method of claim 1, wherein the explosive-bindable quantum dot is configured such that a molecule able to bind with the explosive is linked to a quantum dot surface.

7. The method of claim 1, wherein the molecule able to bind with the explosive comprises an attachment domain that is strongly linked to a nanop article surface at a first side thereof, a functionality domain able to bind with the explosive at a second side thereof, and an intermediate connection domain between the attachment domain and the functionality domain.

8. The method of claim 7, wherein the attachment domain is selected from the group consisting of a dithiol group, a thiol group (—SH), an amine group (—NH₂, —NH), a phosphonate group (—PO₃H), a phosphide group (—P), a phosphine oxide group (—P═O), a carboxyl group (−COOH), a hydroxyl group (−OH), an imidazole group, and a diol group.

9. The method of claim 7, wherein the functionality domain is an amine group, a peptide or an antibody, which is able to bind with the explosive.

10. The method of claim 1, wherein the explosive is a nitroaromatic compound.

11. The method of claim 1, wherein the quantum dot thin film has a fluorescence wavelength longer by at least 50 nm than a quantum dot solution.

12. A sensor for detecting an explosive, comprising:
a light source;
a substrate having an explosive-bindable quantum dot thin film; and
a fluorescence spectrometer for measuring a change in fluorescence of a quantum dot.

13. The sensor of claim 12, wherein the fluorescence of the quantum dot is transferred to the fluorescence spectrometer via an optical fiber.

14. The sensor of claim 12, wherein the substrate is a glass substrate.

15. The sensor of claim 12, further comprising a fluorescence microscope.

16. The sensor of claim 12, wherein the substrate having the quantum dot thin film is obtained by casting a quantum dot solution on a substrate and then performing drying.

17. The sensor of claim 12, wherein the quantum dot has a higher-order amine group on a surface thereof.

18. The sensor of claim 12, wherein the explosive is a nitroaromatic compound.

19. The sensor of claim 12, wherein the quantum dot has a concentration of 0.1˜10 pmol/cm².

20. The sensor of claim 12, wherein the thin film has a thickness of 0.1˜100 μm.
